SEQUENCE LISTING (161 sequences)

SEQ ID NO: 1 - DNA, 20 bp, synthetic construct
  source 1..20: mol_type = other DNA
ataacaacctttccctctgg

SEQ ID NO: 2 - DNA, 20 bp, synthetic construct
  source 1..20: mol_type = other DNA
cctttccctctggagtttta

SEQ ID NO: 3 - DNA, 20 bp, synthetic construct
  source 1..20: mol_type = other DNA
ggaaataaagatgaagcagg

SEQ ID NO: 4 - DNA, 20 bp, synthetic construct
  source 1..20: mol_type = other DNA
gcgtcaaaataagagtcatg

SEQ ID NO: 5 - DNA, 20 bp, synthetic construct
  source 1..20: mol_type = other DNA
ggccatgagttaatgattgt

SEQ ID NO: 6 - DNA, 20 bp, synthetic construct
  source 1..20: mol_type = other DNA
ggtgtatggaatttcccata

SEQ ID NO: 7 - DNA, 20 bp, synthetic construct
  source 1..20: mol_type = other DNA
ctcaccaggggtattgggtg

SEQ ID NO: 8 - DNA, 20 bp, synthetic construct
  source 1..20: mol_type = other DNA
taatcccaagcatttattgc

SEQ ID NO: 9 - DNA, 20 bp, synthetic construct
  source 1..20: mol_type = other DNA
ggaactgtacatgatcagat

SEQ ID NO: 10 - DNA, 20 bp, synthetic construct
  source 1..20: mol_type = other DNA
gatcagatcatggaaggata

SEQ ID NO: 11 - DNA, 20 bp, synthetic construct
  source 1..20: mol_type = other DNA
agatcatggaaggatagctc

SEQ ID NO: 12 - DNA, 20 bp, synthetic construct
  source 1..20: mol_type = other DNA
gtggccactgccatcttcct

SEQ ID NO: 13 - DNA, 20 bp, synthetic construct
  source 1..20: mol_type = other DNA
tcatcactgtccttagcttt

SEQ ID NO: 14 - DNA, 20 bp, synthetic construct
  source 1..20: mol_type = other DNA
tgtccttagctttggagtac

SEQ ID NO: 15 - DNA, 20 bp, synthetic construct
  source 1..20: mol_type = other DNA
gtacacaactccaagtggcc

SEQ ID NO: 16 - DNA, 20 bp, synthetic construct
  source 1..20: mol_type = other DNA
caaattccacactgatagca

SEQ ID NO: 17 - DNA, 20 bp, synthetic construct
  source 1..20: mol_type = other DNA
ccacactgatagcaacaatg

SEQ ID NO: 18 - DNA, 20 bp, synthetic construct
  source 1..20: mol_type = other DNA
gactgcatctgatgtgtgct

SEQ ID NO: 19 - DNA, 20 bp, synthetic construct
  source 1..20: mol_type = other DNA
gctggcaatcttaagcccaa

SEQ ID NO: 20 - DNA, 20 bp, synthetic construct
  source 1..20: mol_type = other DNA
gctgagtgagaaggcatgga

SEQ ID NO: 21 - DNA, 20 bp, synthetic construct
  source 1..20: mol_type = other DNA
gagtgagaaggcatggacta

SEQ ID NO: 22 - DNA, 20 bp, synthetic construct
  source 1..20: mol_type = other DNA
catggactactaaattctgg

SEQ ID NO: 23 - DNA, 20 bp, synthetic construct
  source 1..20: mol_type = other DNA
ttacaaaacaacttcactac

SEQ ID NO: 24 - DNA, 20 bp, synthetic construct
  source 1..20: mol_type = other DNA
cgccatcacaacctttctct

SEQ ID NO: 25 - DNA, 20 bp, synthetic construct
  source 1..20: mol_type = other DNA
ctagcagaaatttttacccc

SEQ ID NO: 26 - DNA, 20 bp, synthetic construct
  source 1..20: mol_type = other DNA
cccatagaatctttcagctt

SEQ ID NO: 27 - DNA, 20 bp, synthetic construct
  source 1..20: mol_type = other DNA
ccagatctatgaccatcctg

SEQ ID NO: 28 - DNA, 20 bp, synthetic construct
  source 1..20: mol_type = other DNA
gatctatgaccatcctgtca

SEQ ID NO: 29 - DNA, 20 bp, synthetic construct
  source 1..20: mol_type = other DNA
ccatcctgtcaatttcctct

SEQ ID NO: 30 - DNA, 20 bp, synthetic construct
  source 1..20: mol_type = other DNA
atgaagaagactattggatg

SEQ ID NO: 31 - DNA, 20 bp, synthetic construct
  source 1..20: mol_type = other DNA
ctcttctccatgtttttctc

SEQ ID NO: 32 - DNA, 20 bp, synthetic construct
  source 1..20: mol_type = other DNA
gttgggaaaaactcttctcc

SEQ ID NO: 33 - DNA, 20 bp, synthetic construct
  source 1..20: mol_type = other DNA
gtttccatgtcaggtttgat

SEQ ID NO: 34 - DNA, 20 bp, synthetic construct
  source 1..20: mol_type = other DNA
ccatgtcaggtttgatctgc

SEQ ID NO: 35 - DNA, 20 bp, synthetic construct
  source 1..20: mol_type = other DNA
gatgatactggcgtaactgg

SEQ ID NO: 36 - DNA, 20 bp, synthetic construct
  source 1..20: mol_type = other DNA
ggcagcagcataatctgaaa

SEQ ID NO: 37 - DNA, 20 bp, synthetic construct
  source 1..20: mol_type = other DNA
ccagcaacttcactttctca

SEQ ID NO: 38 - DNA, 20 bp, synthetic construct
  source 1..20: mol_type = other DNA
gcatcttctgcactgatctc

SEQ ID NO: 39 - DNA, 20 bp, synthetic construct
  source 1..20: mol_type = other DNA
tagtcttctcctcttgccgc

SEQ ID NO: 40 - DNA, 20 bp, synthetic construct
  source 1..20: mol_type = other DNA
ctcatagtcttctcctcttg

SEQ ID NO: 41 - DNA, 20 bp, synthetic construct
  source 1..20: mol_type = other DNA
ttcccacttccggcaacaag

SEQ ID NO: 42 - DNA, 20 bp, synthetic construct
  source 1..20: mol_type = other DNA
ctcttcccacttccggcaac

SEQ ID NO: 43 - DNA, 20 bp, synthetic construct
  source 1..20: mol_type = other DNA
agccgccatcacaacctttc

SEQ ID NO: 44 - DNA, 20 bp, synthetic construct
  source 1..20: mol_type = other DNA
gtatatgcgtcaaaataaga

SEQ ID NO: 45 - DNA, 20 bp, synthetic construct
  source 1..20: mol_type = other DNA
gttagaaattctaatcccaa

SEQ ID NO: 46 - DNA, 20 bp, synthetic construct
  source 1..20: mol_type = other DNA
tacatcttatatccaagcac

SEQ ID NO: 47 - DNA, 20 bp, synthetic construct
  source 1..20: mol_type = other DNA
ggattagacagctgttcctc

SEQ ID NO: 48 - DNA, 20 bp, synthetic construct
  source 1..20: mol_type = other DNA
gatatctgcatcatcattat

SEQ ID NO: 49 - DNA, 20 bp, synthetic construct
  source 1..20: mol_type = other DNA
tctgcatcatcattataagg

SEQ ID NO: 50 - DNA, 20 bp, synthetic construct
  source 1..20: mol_type = other DNA
gtaactgggcagcagcataa

SEQ ID NO: 51 - DNA, 20 bp, synthetic construct
  source 1..20: mol_type = other DNA
taacaacctttccctctgga

SEQ ID NO: 52 - DNA, 20 bp, synthetic construct
  source 1..20: mol_type = other DNA
aggcatggactactaaattc

SEQ ID NO: 53 - DNA, 20 bp, synthetic construct
  source 1..20: mol_type = other DNA
gaaggcatggactactaaat

SEQ ID NO: 54 - DNA, 20 bp, synthetic construct
  source 1..20: mol_type = other DNA
ctgagtgagaaggcatggac

SEQ ID NO: 55 - DNA, 20 bp, synthetic construct
  source 1..20: mol_type = other DNA
tgagtgagaaggcatggact

SEQ ID NO: 56 - DNA, 20 bp, synthetic construct
  source 1..20: mol_type = other DNA
agaaggcatggactactaaa

SEQ ID NO: 57 - DNA, 20 bp, synthetic construct
  source 1..20: mol_type = other DNA
gagaaggcatggactactaa

SEQ ID NO: 58 - DNA, 20 bp, synthetic construct
  source 1..20: mol_type = other DNA
tgagaaggcatggactacta

SEQ ID NO: 59 - DNA, 20 bp, synthetic construct
  source 1..20: mol_type = other DNA
gtgagaaggcatggactact

SEQ ID NO: 60 - DNA, 20 bp, synthetic construct
  source 1..20: mol_type = other DNA
agtgagaaggcatggactac

SEQ ID NO: 61 - DNA, 20 bp, synthetic construct
  source 1..20: mol_type = other DNA
aatctttcagctttcttgtt

SEQ ID NO: 62 - DNA, 20 bp, synthetic construct
  source 1..20: mol_type = other DNA
agaatctttcagctttcttg

SEQ ID NO: 63 - DNA, 20 bp, synthetic construct
  source 1..20: mol_type = other DNA
ctactaaattctggattact

SEQ ID NO: 64 - DNA, 20 bp, synthetic construct
  source 1..20: mol_type = other DNA
cacactgatagcaacaatga

SEQ ID NO: 65 - DNA, 20 bp, synthetic construct
  source 1..20: mol_type = other DNA
acactgatagcaacaatgac

SEQ ID NO: 66 - DNA, 20 bp, synthetic construct
  source 1..20: mol_type = other DNA
cactgatagcaacaatgact

SEQ ID NO: 67 - DNA, 20 bp, synthetic construct
  source 1..20: mol_type = other DNA
actgatagcaacaatgactg

SEQ ID NO: 68 - DNA, 20 bp, synthetic construct
  source 1..20: mol_type = other DNA
ctgatagcaacaatgactgc

SEQ ID NO: 69 - DNA, 20 bp, synthetic construct
  source 1..20: mol_type = other DNA
tgatagcaacaatgactgca

SEQ ID NO: 70 - DNA, 20 bp, synthetic construct
  source 1..20: mol_type = other DNA
ggcaatcttaagcccaaaat

SEQ ID NO: 71 - DNA, 20 bp, synthetic construct
  source 1..20: mol_type = other DNA
gcaatcttaagcccaaaatg

SEQ ID NO: 72 - DNA, 20 bp, synthetic construct
  source 1..20: mol_type = other DNA
caatcttaagcccaaaatgc

SEQ ID NO: 73 - DNA, 20 bp, synthetic construct
  source 1..20: mol_type = other DNA
atgcttcaaagattaaacag

SEQ ID NO: 74 - DNA, 20 bp, synthetic construct
  source 1..20: mol_type = other DNA
cttcaaagattaaacagcca

SEQ ID NO: 75 - DNA, 20 bp, synthetic construct
  source 1..20: mol_type = other DNA
catcttatatccaagcacaa

SEQ ID NO: 76 - DNA, 20 bp, synthetic construct
  source 1..20: mol_type = other DNA
atcttatatccaagcacaaa

SEQ ID NO: 77 - DNA, 20 bp, synthetic construct
  source 1..20: mol_type = other DNA
cttatatccaagcacaaaaa

SEQ ID NO: 78 - DNA, 20 bp, synthetic construct
  source 1..20: mol_type = other DNA
catgaaaggatatacgttta

SEQ ID NO: 79 - DNA, 20 bp, synthetic construct
  source 1..20: mol_type = other DNA
atttctaaatgctgagtgag

SEQ ID NO: 80 - DNA, 20 bp, synthetic construct
  source 1..20: mol_type = other DNA
ctaaatgctgagtgagaagg

SEQ ID NO: 81 - DNA, 20 bp, synthetic construct
  source 1..20: mol_type = other DNA
taaatgctgagtgagaaggc

SEQ ID NO: 82 - DNA, 20 bp, synthetic construct
  source 1..20: mol_type = other DNA
aaatgctgagtgagaaggca

SEQ ID NO: 83 - DNA, 20 bp, synthetic construct
  source 1..20: mol_type = other DNA
aatgctgagtgagaaggcat

SEQ ID NO: 84 - DNA, 20 bp, synthetic construct
  source 1..20: mol_type = other DNA
atgctgagtgagaaggcatg

SEQ ID NO: 85 - DNA, 20 bp, synthetic construct
  source 1..20: mol_type = other DNA
tgctgagtgagaaggcatgg

SEQ ID NO: 86 - DNA, 20 bp, synthetic construct
  source 1..20: mol_type = other DNA
catgaagaagactattggat

SEQ ID NO: 87 - DNA, 20 bp, synthetic construct
  source 1..20: mol_type = other DNA
tacacaactccaagtggccc

SEQ ID NO: 88 - DNA, 20 bp, synthetic construct
  source 1..20: mol_type = other DNA
acacaactccaagtggcccg

SEQ ID NO: 89 - DNA, 20 bp, synthetic construct
  source 1..20: mol_type = other DNA
catcactgtccttagctttg

SEQ ID NO: 90 - DNA, 20 bp, synthetic construct
  source 1..20: mol_type = other DNA
atcactgtccttagctttgg

SEQ ID NO: 91 - DNA, 20 bp, synthetic construct
  source 1..20: mol_type = other DNA
tcactgtccttagctttgga

SEQ ID NO: 92 - DNA, 20 bp, synthetic construct
  source 1..20: mol_type = other DNA
cactgtccttagctttggag

SEQ ID NO: 93 - DNA, 20 bp, synthetic construct
  source 1..20: mol_type = other DNA
actgtccttagctttggagt

SEQ ID NO: 94 - DNA, 20 bp, synthetic construct
  source 1..20: mol_type = other DNA
ctgtccttagctttggagta

SEQ ID NO: 95 - DNA, 20 bp, synthetic construct
  source 1..20: mol_type = other DNA
gatcatggaaggatagctcc

SEQ ID NO: 96 - DNA, 20 bp, synthetic construct
  source 1..20: mol_type = other DNA
atcatggaaggatagctcca

SEQ ID NO: 97 - DNA, 20 bp, synthetic construct
  source 1..20: mol_type = other DNA
tcatggaaggatagctccac

SEQ ID NO: 98 - DNA, 20 bp, synthetic construct
  source 1..20: mol_type = other DNA
ctccccgaccttggtcacag

SEQ ID NO: 99 - DNA, 20 bp, synthetic construct
  source 1..20: mol_type = other DNA
atggaaggatagctccactc

SEQ ID NO: 100 - DNA, 20 bp, synthetic construct
  source 1..20: mol_type = other DNA
ggaaggatagctccactcct

SEQ ID NO: 101 - DNA, 20 bp, synthetic construct
  source 1..20: mol_type = other DNA
cagcccagtcaataaggttc

SEQ ID NO: 102 - DNA, 20 bp, synthetic construct
  source 1..20: mol_type = other DNA
gcctctattgagttgtgtgg

SEQ ID NO: 103 - DNA, 20 bp, synthetic construct
  source 1..20: mol_type = other DNA
tatcaactggctgaactacc

SEQ ID NO: 104 - DNA, 20 bp, synthetic construct
  source 1..20: mol_type = other DNA
atcaactggctgaactacca

SEQ ID NO: 105 - DNA, 20 bp, synthetic construct
  source 1..20: mol_type = other DNA
gtcaggtttgatctgcttgg

SEQ ID NO: 106 - DNA, 20 bp, synthetic construct
  source 1..20: mol_type = other DNA
aaccccagcactaaagcatt

SEQ ID NO: 107 - DNA, 20 bp, synthetic construct
  source 1..20: mol_type = other DNA
ggactcttatccctaataca

SEQ ID NO: 108 - DNA, 20 bp, synthetic construct
  source 1..20: mol_type = other DNA
tccctatcaactggctgaac

SEQ ID NO: 109 - DNA, 20 bp, synthetic construct
  source 1..20: mol_type = other DNA
gctggactcttatccctaat

SEQ ID NO: 110 - DNA, 20 bp, synthetic construct
  source 1..20: mol_type = other DNA
acagtagtttaggtgtcacc

SEQ ID NO: 111 - DNA, 20 bp, synthetic construct
  source 1..20: mol_type = other DNA
gactaacagcccagtcaata

SEQ ID NO: 112 - DNA, 20 bp, synthetic construct
  source 1..20: mol_type = other DNA
actacatgaggcaccccctg

SEQ ID NO: 113 - DNA, 20 bp, synthetic construct
  source 1..20: mol_type = other DNA
gagaggctccaggacttatc

SEQ ID NO: 114 - DNA, 20 bp, synthetic construct
  source 1..20: mol_type = other DNA
ttgctggactcttatcccta

SEQ ID NO: 115 - DNA, 20 bp, synthetic construct
  source 1..20: mol_type = other DNA
ggttgttagtaatggagact

SEQ ID NO: 116 - DNA, 20 bp, synthetic construct
  source 1..20: mol_type = other DNA
ggtttgatctgcttggtcaa

SEQ ID NO: 117 - DNA, 20 bp, synthetic construct
  source 1..20: mol_type = other DNA
tgtttggaccttcctattga

SEQ ID NO: 118 - DNA, 20 bp, synthetic construct
  source 1..20: mol_type = other DNA
ttaggtcataagctggtggc

SEQ ID NO: 119 - DNA, 20 bp, synthetic construct
  source 1..20: mol_type = other DNA
atccctagtatattcttggc

SEQ ID NO: 120 - DNA, 20 bp, synthetic construct
  source 1..20: mol_type = other DNA
ctaccatccctagtatattc

SEQ ID NO: 121 - DNA, 20 bp, synthetic construct
  source 1..20: mol_type = other DNA
ggtcaggcaggacatattaa

SEQ ID NO: 122 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified thymine
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified thymine
  modified_base 17: mod_base = OTHER; note = 2'-methoxyethyl modified nucleotide
  modified_base 18: mod_base = OTHER; note = 2'-methoxyethyl modified thymine with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
ataacaacctttccctctgg

SEQ ID NO: 123 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified thymine
  modified_base 4: mod_base = OTHER; note = 2'-methoxyethyl modified thymine
  modified_base 5: mod_base = OTHER; note = 2'-methoxyethyl modified thymine
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified thymine
  modified_base 17: mod_base = OTHER; note = 2'-methoxyethyl modified thymine
  modified_base 18: mod_base = OTHER; note = 2'-methoxyethyl modified thymine with phosphorothioate linkage
  modified_base 19: mod_base = OTHER; note = 2'-methoxyethyl modified thymine with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
cctttccctctggagtttta

SEQ ID NO: 124 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
ggaaataaagatgaagcagg

SEQ ID NO: 125 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified thymine
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified thymine
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified thymine with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
gcgtcaaaataagagtcatg

SEQ ID NO: 126 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified thymine
  modified_base 18: mod_base = OTHER; note = 2'-methoxyethyl modified thymine with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified thymine
  misc_feature 1..5: note = RNA
  misc_feature 6..15: note = DNA
  misc_feature 16..20: note = RNA
ggccatgagttaatgattgt

SEQ ID NO: 127 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified thymine
  modified_base 4: mod_base = OTHER; note = 2'-methoxyethyl modified nucleotide
  modified_base 5: mod_base = OTHER; note = 2'-methoxyethyl modified thymine
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified thymine with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
ggtgtatggaatttcccata

SEQ ID NO: 128 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified thymine
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified thymine with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
ctcaccaggggtattgggtg

SEQ ID NO: 129 - DNA, 20 bp, synthetic construct
  source 1..20: mol_type = other DNA
  modified_base 1: mod_base = OTHER; note = 2'-methoxyethyl modified thymine with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified thymine
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified thymine
  modified_base 18: mod_base = OTHER; note = 2'-methoxyethyl modified thymine with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
taatcccaagcatttattgc

SEQ ID NO: 130 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified thymine
  misc_feature 1..5: note = RNA
  misc_feature 6..15: note = DNA
  misc_feature 16..20: note = RNA
ggaactgtacatgatcagat

SEQ ID NO: 131 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified thymine
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified thymine with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
gatcagatcatggaaggata

SEQ ID NO: 132 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified thymine
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified thymine with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
agatcatggaaggatagctc

SEQ ID NO: 133 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified thymine
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified thymine
  modified_base 17: mod_base = OTHER; note = 2'-methoxyethyl modified thymine
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified thymine
  misc_feature 1..5: note = RNA
  misc_feature 6..15: note = DNA
  misc_feature 16..20: note = RNA
gtggccactgccatcttcct

SEQ ID NO: 134 - DNA, 20 bp, synthetic construct
  source 1..20: mol_type = other DNA
  modified_base 1: mod_base = OTHER; note = 2'-methoxyethyl modified thymine with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified thymine
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified nucleotide
  modified_base 18: mod_base = OTHER; note = 2'-methoxyethyl modified thymine with phosphorothioate linkage
  modified_base 19: mod_base = OTHER; note = 2'-methoxyethyl modified thymine with phosphorothioate linkage
  modified_base 20: mod_base = OTHER; note = 2'-methoxyethyl modified thymine
  misc_feature 1..5: note = RNA
  misc_feature 6..15: note = DNA
  misc_feature 16..20: note = RNA
tcatcactgtccttagcttt

SEQ ID NO: 135 - DNA, 20 bp, synthetic construct
  source 1..20: mol_type = other DNA
  modified_base 1: mod_base = OTHER; note = 2'-methoxyethyl modified thymine with phosphorothioate linkage
  modified_base 2: mod_base = OTHER; note = 2'-methoxyethyl modified nucleotide
  modified_base 3: mod_base = OTHER; note = 2'-methoxyethyl modified thymine
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified nucleotide
  modified_base 18: mod_base = OTHER; note = 2'-methoxyethyl modified thymine with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
tgtccttagctttggagtac

SEQ ID NO: 136 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified thymine
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified thymine
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
gtacacaactccaagtggcc

SEQ ID NO: 137 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified thymine
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified thymine
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
caaattccacactgatagca

SEQ ID NO: 138 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified thymine with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
ccacactgatagcaacaatg

SEQ ID NO: 139 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified thymine
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified thymine
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified thymine
  misc_feature 1..5: note = RNA
  misc_feature 6..15: note = DNA
  misc_feature 16..20: note = RNA
gactgcatctgatgtgtgct

SEQ ID NO: 140 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified thymine
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
gctggcaatcttaagcccaa

SEQ ID NO: 141 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified thymine
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified thymine
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
gctgagtgagaaggcatgga

SEQ ID NO: 142 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified thymine
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified thymine
  modified_base 17: mod_base = OTHER; note = 2'-methoxyethyl modified nucleotide
  modified_base 18: mod_base = OTHER; note = 2'-methoxyethyl modified thymine with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
gcctctattgagttgtgtgg

SEQ ID NO: 143 - DNA, 20 bp, synthetic construct
  source 1..20: mol_type = other DNA
  modified_base 1: mod_base = OTHER; note = 2'-methoxyethyl modified thymine with phosphorothioate linkage
  modified_base 2: mod_base = OTHER; note = 2'-methoxyethyl modified nucleotide
  modified_base 3: mod_base = OTHER; note = 2'-methoxyethyl modified thymine
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified thymine
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
tatcaactggctgaactacc

SEQ ID NO: 144 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified thymine
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified thymine
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
atcaactggctgaactacca

SEQ ID NO: 145 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified thymine
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified thymine
  modified_base 18: mod_base = OTHER; note = 2'-methoxyethyl modified thymine with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
gtcaggtttgatctgcttgg

SEQ ID NO: 146 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified thymine with phosphorothioate linkage
  modified_base 20: mod_base = OTHER; note = 2'-methoxyethyl modified thymine
  misc_feature 1..5: note = RNA
  misc_feature 6..15: note = DNA
  misc_feature 16..20: note = RNA
aaccccagcactaaagcatt

SEQ ID NO: 147 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified thymine
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified thymine
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
ggactcttatccctaataca

SEQ ID NO: 148 - DNA, 20 bp, synthetic construct
  source 1..20: mol_type = other DNA
  modified_base 1: mod_base = OTHER; note = 2'-methoxyethyl modified thymine with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified thymine
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified thymine
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
tccctatcaactggctgaac

SEQ ID NO: 149 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified thymine
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified thymine
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified thymine
  misc_feature 1..5: note = RNA
  misc_feature 6..15: note = DNA
  misc_feature 16..20: note = RNA
gctggactcttatccctaat

SEQ ID NO: 150 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified thymine
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified thymine
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
acagtagtttaggtgtcacc

SEQ ID NO: 151 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified thymine
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified thymine with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
gactaacagcccagtcaata

SEQ ID NO: 152 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified thymine
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified thymine with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
actacatgaggcaccccctg

SEQ ID NO: 153 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified thymine
  modified_base 17: mod_base = OTHER; note = 2'-methoxyethyl modified thymine
  modified_base 18: mod_base = OTHER; note = 2'-methoxyethyl modified nucleotide with phosphorothioate linkage
  modified_base 19: mod_base = OTHER; note = 2'-methoxyethyl modified thymine with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
gagaggctccaggacttatc

SEQ ID NO: 154 - DNA, 20 bp, synthetic construct
  source 1..20: mol_type = other DNA
  modified_base 1: mod_base = OTHER; note = 2'-methoxyethyl modified thymine with phosphorothioate linkage
  modified_base 2: mod_base = OTHER; note = 2'-methoxyethyl modified thymine
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified thymine
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified thymine with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
ttgctggactcttatcccta

SEQ ID NO: 155 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified thymine
  modified_base 4: mod_base = OTHER; note = 2'-methoxyethyl modified thymine
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified nucleotide
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified thymine
  misc_feature 1..5: note = RNA
  misc_feature 6..15: note = DNA
  misc_feature 16..20: note = RNA
ggttgttagtaatggagact

SEQ ID NO: 156 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified thymine
  modified_base 4: mod_base = OTHER; note = 2'-methoxyethyl modified thymine
  modified_base 5: mod_base = OTHER; note = 2'-methoxyethyl modified thymine
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified thymine
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
ggtttgatctgcttggtcaa

SEQ ID NO: 157 - DNA, 20 bp, synthetic construct
  source 1..20: mol_type = other DNA
  modified_base 1: mod_base = OTHER; note = 2'-methoxyethyl modified thymine with phosphorothioate linkage
  modified_base 2: mod_base = OTHER; note = 2'-methoxyethyl modified nucleotide
  modified_base 3: mod_base = OTHER; note = 2'-methoxyethyl modified thymine
  modified_base 4: mod_base = OTHER; note = 2'-methoxyethyl modified thymine
  modified_base 5: mod_base = OTHER; note = 2'-methoxyethyl modified thymine
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified thymine
  modified_base 18: mod_base = OTHER; note = 2'-methoxyethyl modified thymine with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
tgtttggaccttcctattga

SEQ ID NO: 158 - DNA, 20 bp, synthetic construct
  source 1..20: mol_type = other DNA
  modified_base 1: mod_base = OTHER; note = 2'-methoxyethyl modified thymine with phosphorothioate linkage
  modified_base 2: mod_base = OTHER; note = 2'-methoxyethyl modified thymine
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified thymine
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
ttaggtcataagctggtggc

SEQ ID NO: 159 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified thymine
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified thymine
  modified_base 17: mod_base = OTHER; note = 2'-methoxyethyl modified thymine
  modified_base 18: mod_base = OTHER; note = 2'-methoxyethyl modified nucleotide with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
atccctagtatattcttggc

SEQ ID NO: 160 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified thymine
  modified_base 3: mod_base = OTHER; note = 2'-methoxyethyl modified nucleotide
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified thymine
  modified_base 17: mod_base = OTHER; note = 2'-methoxyethyl modified nucleotide
  modified_base 18: mod_base = OTHER; note = 2'-methoxyethyl modified thymine with phosphorothioate linkage
  modified_base 19: mod_base = OTHER; note = 2'-methoxyethyl modified thymine with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
ctaccatccctagtatattc

SEQ ID NO: 161 - DNA, 20 bp, synthetic construct
  source 1..20: mol_type = other DNA
  modified_base 1: mod_base = OTHER; note = 2'-methoxyethyl modified nucleotide with phosphorothioate linkage
  modified_base 2: mod_base = OTHER; note = 2'-methoxyethyl modified nucleotide
  modified_base 3: mod_base = OTHER; note = 2'-methoxyethyl modified thymine
  modified_base 4: mod_base = OTHER; note = 2'-methoxyethyl modified nucleotide
  modified_base 5: mod_base = OTHER; note = 2'-methoxyethyl modified nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-methoxyethyl modified nucleotide
  modified_base 17: mod_base = OTHER; note = 2'-methoxyethyl modified thymine
  modified_base 18: mod_base = OTHER; note = 2'-methoxyethyl modified thymine with phosphorothioate linkage
  modified_base 19: mod_base = OTHER; note = 2'-methoxyethyl modified nucleotide with phosphorothioate linkage
  modified_base 20: mod_base = OTHER; note = 2'-methoxyethyl modified nucleotide
  misc_feature 1..5: note = RNA
  misc_feature 6..15: note = DNA
  misc_feature 16..20: note = RNA
ggtcaggcaggacatattaa

(19) United States
(12) Patent Application Publication
(10) Pub. No.: US 2025/0223591 A1
(43) Pub. Date: Jul. 10, 2025

(54) SYF2 ANTISENSE OLIGONUCLEOTIDES

(71) Applicant: AcuraStem Incorported, Pasadena, CA (US)

(72) Inventors: Emily Elizabeth LEE, Pasadena, CA (US); Wen-Hsuan CHANG, Pasadena, CA (US)

(21) Appl. No.: 18/860,677

(22) PCT Filed: Apr. 27, 2023
(86) PCT No.: PCT/US2023/066274

§ 371 Date: Oct. 27, 2024

Related U.S. Application Data
(60) Provisional application No. 63/363,729, filed on Apr. 28, 2022.

(51) Int. Cl. C12N 15/113; A61P 25/28

(52) U.S. Cl. CPC C12N 15/113; A61P 25/28; C12N 2310/11; C12N 2310/315; C12N 2310/321; C12N 2310/322; C12N 2310/3341; C12N 2310/341

(57) ABSTRACT

The present invention relates to SYF2 antisense oligonucleotides (ASOs), pharmaceutical compositions containing them, and methods for treating, inhibiting, suppressing, and preventing neurological diseases with them.

### CROSS-REFERENCE TO RELATED APPLICATIONS

The present application is the U.S. national stage of International Patent Application No. PCT/US2023/066274, filed Apr. 27, 2023, which claims the benefit of U.S. Provisional Application No. 63/363,729 filed Apr. 28, 2022, which are hereby incorporated by reference in its entirety.

### FIELD OF THE INVENTION

The present invention relates to SYF2 antisense oligonucleotides (ASOs), pharmaceutical compositions containing them, and methods for treating, inhibiting, suppressing, and preventing neurological or neurodegenerative diseases with them.

### BACKGROUND OF THE INVENTION

Many neurodegenerative disorders in patients are difficult to effectively treat, especially where the pathology of a neurodegenerative disorder in a particular patient is not completely understood.

International Publication No. WO 2021/150840 discloses a method of treating a neurodegenerative disease by administering a SYF2 inhibitor. SYF2 binds to pre-mRNA splicing complexes and although it is not essential for splicing, evidence suggests it regulates nuclear export of mRNA.

There remains a need for effective treatments for many neurodegenerative disorders, such as amyotrophic lateral sclerosis (ALS) and frontotemporal dementia (FTD).

### SUMMARY OF THE INVENTION

The present invention relates to SYF2 antisense oligonucleotides (ASOs), pharmaceutical compositions containing them, and their use in the treatment of neurodegenerative disorders.

One embodiment is a single stranded ASO that suppresses the expression of SYF2, wherein the ASO has a nucleobase sequence that comprises at least 12 or 15 consecutive nucleobases of any of the nucleobase sequences of SEQ ID NOs: 1-121. The nucleobase sequence of the ASO can comprise up to 30, 25, 24, 23, 22, 21, or 20 consecutive nucleobases of any of the nucleobase sequences of SEQ ID NOs: 1-121. The ASO can also be any of SEQ ID NOs: 1-121.

Another embodiment is an oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or at least 20 consecutive nucleobases of any of the nucleobase sequences of SEQ ID NOs: 1-121. The oligonucleotide can comprise up to 25, 24, 23, 22, 21, or 20 consecutive nucleobases of any of the nucleobase sequences of SEQ ID NOs: 1-121.

In certain embodiments, at least one internucleoside linkage is a modified internucleoside linkage, and the modified internucleoside linkage may be a phosphorothioate internucleoside linkage or a phosphodiester internucleoside linkage. At least one of the nucleosides may also be a modified nucleobase.

In other embodiments, at least one nucleoside of the ASO may be a modified sugar moiety, where that modified sugar moiety can be a bicyclic sugar moiety, or the modified sugar moiety may comprise a 2′-O-methoxyethyl group, 2′-F, or 2′-O-hexadecyl group. In certain aspects, the bicyclic sugar moiety comprises a 4′-CH(R)—O-2′ bridge where the R group is, independently, H, C₁₋₁₂ alkyl, or a protecting group. In one embodiment, the bicyclic sugar moiety comprises a 4-(CH₂)₂—O-2 bridge (“ENA” or ethylene-bridged nucleic acid).

In yet other embodiments, the ASO is a gapmer (e.g., a MOE gapmer), where a gap segment may consist of 8 to 12 linked deoxynucleosides, a 5′ wing segment consisting of 3 to 5 linked nucleosides, and a 3′ wing segment consisting of 3 to 5 linked nucleosides. In certain aspects, the gap segment may be positioned between the 5′ wing segment and the 3′ wing segment, where a nucleoside of each wing segment comprises a modified sugar moiety (e.g., one with a 2′-O-methoxyethyl group).

In other embodiments, the oligonucleotide consists of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or at least 20 consecutive nucleobases of any of the nucleobase sequences of SEQ ID NOs: 1-121.

Another embodiment is a pharmaceutical composition comprising a SYF2 ASO of the present invention and one or more pharmaceutically acceptable carriers, diluents, and/or excipients. In one embodiment, the pharmaceutical composition is suitable for parenteral administration, such as intracerebroventricular injection or intrathecal administration.

Yet another embodiment is a method of treating a subject having a neurological or neurodegenerative disease by administering a therapeutically effective amount of a SYF2 ASO or a pharmaceutical composition described herein.

Yet another embodiment is a method of treating a subject having a SYF2 disease or disorder by administering a therapeutically effective amount of a SYF2 ASO or a pharmaceutical composition described herein.

### BRIEF DESCRIPTION OF THE FIGURES

A more complete appreciation of the invention and many of the attendant advantages thereof will be readily obtained as the same becomes better understood by reference to the following detailed description when considered in connection with the accompanying figures, wherein:

FIG. 1A shows a SYF2 ASO screen in HeLa cells, measuring the fold change normalized to GadpH of ASO 51-74 (SEQ ID NOs. 51-74) against a control (NCASO), ASO 20 (SEQ ID NO: 20), and ASO 21 (SEQ ID NO: 21).

FIG. 1B shows a SYF2 ASO screen in HeLa cells, measuring the fold change normalized to GadpH of ASO 75-100 (SEQ ID NOs. 75-100) against a control (NCASO), ASO 20 (SEQ ID NO: 20), and ASO 21 (SEQ ID NO: 21).

FIG. 1C shows a SYF2 ASO screen in HeLa cells, measuring the fold change normalized to GadpH of ASO 101-121 (SEQ ID NOs. 101-121) against a control (NCASO) and ASO 21 (SEQ ID NO: 21).

### DETAILED DESCRIPTION OF THE INVENTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting. The terminology used herein is for the purpose of describing particular embodiments only and is not intended to be limiting.

### Definitions

The terms “comprise(s),” “include(s),” “having,” “has,” “can,” “contain(s),” “may” and variants thereof, as used herein, are intended to be open-ended transitional phrases, terms, or words that do not preclude the possibility of additional acts or structures.

The singular forms “a,” “an” and “the” include plural references unless the context clearly dictates otherwise.

The present disclosure also contemplates other embodiments “comprising,” “consisting of” and “consisting essentially of,” the embodiments or elements presented herein, whether explicitly set forth or not.

As used herein, “2′-deoxynucleoside” means a nucleoside comprising a 2′-H(H) furanosyl sugar moiety, as found in naturally occurring deoxyribonucleic acids (DNA) and a nucleobase. In certain embodiments, a 2′-deoxynucleoside may comprise a modified nucleobase and a furanosyl sugar moiety or may comprise an RNA nucleobase (uracil) and a furanosyl sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “antisense molecule” means an oligomeric nucleic acid or oligomeric duplex capable of achieving at least one antisense activity.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety. As used herein, “bicyclic nucleoside” or “BNA” means a nucleoside comprising a bicyclic sugar moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T); adenine (A) and uracil (U); cytosine (C) and guanine (G); and 5-methylcytosine (mC) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” Unless otherwise indicated, “gapmer” refers to a sugar motif. Unless otherwise indicated, the sugar moieties of the nucleosides of the gap of a gapmer are unmodified 2′-deoxyfuranosyl. Thus, the term “MOE gapmer” indicates a gapmer having a sugar motif of 2′-MOE nucleosides in both wings and a gap of 2′-deoxynucleosides. Unless otherwise indicated, a MOE gapmer may comprise one or more modified internucleoside linkages and/or modified nucleobases and such modifications do not necessarily follow the gapmer pattern of the sugar modifications. Table 2, below, provides exemplary MOE-gapmers.

“Inhibit” as used herein refers to the ability to substantially antagonize, prohibit, prevent, suppress, restrain, slow, disrupt, alter, eliminate, stop, or reverse the progression or severity of the activity of a particular agent (e.g., infectious agent) or disease.

As used herein, the term “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate linkage” is a modified internucleoside linkage in which one of the non-bridging oxygen atoms of a phosphodiester internucleoside linkage is replaced with a sulfur atom.

As used herein, “MOE” means methoxyethyl. “2′-MOE” means a —OCH₂CH₂OCH₃ group at the 2′ position of a furanosyl ring.

A “neurological disease” is any disease that causes electrical, biochemical, or structural abnormalities in the brain, spine, or neurons. For example, a neurological disease may be a neurodegenerative disease.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. As used herein, an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase or modified nucleobase. A “5-methylcytosine” or “mC” is a modified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a continuous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. The internucleoside linkages may be any described herein. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

“SYF2”, also known in the art as “SYF2 Pre-MRNA Splicing Factor”, encodes a nuclear protein that interacts with cyclin D-type binding-protein 1, which is thought to be a cell cycle regulator at the G1/S transition. SYF2 has been shown to reduce TDP-43 aggregation and mislocalization and cryptic exon inclusion, while also rescuing C9ORF72 and ensuring sporadic ALS neuron survival. TDP-43 has been shown to decrease the incorporation of cryptic exons that result in non-productive RNA transcripts. Consequently, a loss or decrease in nuclear TDP-43 in motor neurons will result in a reduction of mRNA levels for many genes, including STWN2, which transcribes a microtubule-binding protein. The loss of STMN2 has been shown to compromise neurite outgrowth in motor neurons.

As used herein a “SYF2 disease or disorder” includes lysosomal degradation diseases and disorders mediated by SYF2. For example, the a SYF2 disease or disorder includes, but is not limited to, amyloid diseases (such as Alzheimer's disease, Parkinson's disease, Huntington's disease, type 2 diabetes, diabetic amyloidosis and chronic hemodialysis-related amyloid), multiple sclerosis, and an MPS disorder (such as MPS I, MPS II, MPS IIIA, MPS IIIB, MPS IIIC, MPS HID, MPS IVA, MPS IVB, MPS VI, MPS VII, or MPS IX). In some embodiments, the diseases are autoimmune disorders (such as multiple sclerosis, rheumatoid arthritis, juvenile chronic arthritis, Ankylosing spondylitis, psoriasis, psoriatic arthritis, adult still disease, Becet syndrome, familial Mediterranean fever, Crohn's disease, leprosy, osteomyelitis, tuberculosis, chronic bronchiectasis, Castleman disease), or CNS disorders (such as spongiform encephalopathies (Creutzfeld-Jakob, Kuru, Mad Cow)). The compositions and methods of the disclosure can be used to treat individuals with lysosomal storage diseases comprising administering to a subject in need of treatment a therapeutically effective amount of a SYF2 ASO or pharmaceutical composition described herein. In some embodiments, the ASOs and compositions of the disclosure decrease or inhibit the activity of SYF2 and alters the biogenesis, function or dynamics of the endosomal or lysosomal systems in a way that reduces the abundance of the material abnormally stored in the lysosome in lysosomal storage diseases. In some embodiments, the ASOs and compositions target, decrease or inhibit the activity of SYF2 thus altering the biogenesis, functions, or dynamics of the endoplasmic reticulum or Golgi apparatus in a way that reduces the abundance of the material abnormally stored in the lysosome in lysosomal storage diseases. In other embodiments, the disease is a neurological disorder.

As used herein, “sugar moiety” means an unmodified sugar moiety or a modified sugar moiety. The superscript prime symbol (′) is used to describe the numbering of a sugar in a nucleoside or nucleotide (the nucleobase positions are numbered without the prime). When describing the sugar only, the prime symbol is not used. As used herein, “unmodified sugar moiety” means a 2-OH(H) furanosyl moiety, as found in RNA (an “unmodified RNA sugar moiety”), or a 2-H(H) moiety, as found in DNA (an “unmodified DNA sugar moiety”). Unmodified sugar moieties have one hydrogen at each of the 1, 3, and 4 positions, an oxygen at the 3 position, and two hydrogens at the 5 position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2-substituted sugar moiety. Such modified furanosyl sugar moieties include bicyclic sugars and nonbicyclic sugars.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. In a preferred embodiment, the subject or patient is a human. The subject or patient may be undergoing other forms of treatment.

A “therapeutically effective amount,” or “effective dosage” or “effective amount” as used interchangeably herein unless otherwise defined, means a dosage of a drug effective for periods of time necessary, to achieve the desired therapeutic result. An effective dosage may be determined by a person skilled in the art and may vary according to factors such as the disease state, age, sex, and weight of the individual, and the ability of the drug to elicit a desired response in the individual. This term as used herein may also refer to an amount effective at bringing about a desired in vivo effect in an animal, mammal, or human, such as reducing and/or inhibiting the function of a receptor. A therapeutically effective amount may be administered in one or more administrations (e.g., the agent may be given as a preventative treatment or therapeutically at any stage of disease progression, before or after symptoms, and the like), applications or dosages and is not intended to be limited to a particular formulation, combination or administration route. It is within the scope of the present disclosure that the drug may be administered at various times during the course of treatment of the subject. The times of administration and dosages used will depend on several factors, such as the goal of treatment (e.g., treating v. preventing), condition of the subject, etc. and can be readily determined by one skilled in the art.

As used herein, the term “treat” or “treating” a subject, refers to administering a composition or agent described herein to the subject, such that at least one symptom of a disease or disorder is healed, alleviated, relieved, altered, remedied, reduced, ameliorated, or improved. Treating includes administering an amount effective to alleviate, relieve, alter, remedy, reduce, ameliorate, and/or improve one or more symptoms associated with a disease or disorder. The treatment may inhibit deterioration or worsening of a symptom associated with the disease or disorder.

### Antisense Oligonucleotides

In certain embodiments, oligonucleotides comprise one or more type of modified sugar and/or unmodified sugar moiety arranged along the oligonucleotide or region thereof in a defined pattern or sugar motif. In certain instances, such sugar motifs include, but are not limited to, any of the sugar modifications discussed herein.

In certain embodiments, modified oligonucleotides comprise or consist of a region having a gapmer motif, which is defined by two external regions or “wings” and a central or internal region or “gap.” The three regions of a gapmer motif include the “5′ wing”, the “gap” and the “3′ wing” which form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, each nucleoside of each wing of a gapmer is a modified nucleoside.

In certain embodiments, the gap of a gapmer comprises 7-12 nucleosides (e.g., 10 nucleosides). In certain embodiments, each nucleoside of the gap of a gapmer is an unmodified 2′-deoxy nucleoside.

In certain embodiments, the gapmer is a deoxy gapmer. In embodiments, the nucleosides on the gap side of each wing/gap junction are unmodified 2′-deoxy nucleosides and the nucleosides on the wing sides of each wing/gap junction are modified nucleosides. In certain embodiments, each nucleoside of the gap is an unmodified 2′-deoxy nucleoside. In certain embodiments, each nucleoside of each wing of a gapmer is a modified nucleoside.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified oligonucleotide comprises the same 2′-modification.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include, but are not limited to, phosphates, which contain a phosphodiester bond (also referred to as unmodified or naturally occurring linkages), phosphotriesters, methylphosphonates or other alkylphosphonates, phosphoramidates, and phosphorothioates, and phosphorodithioates. Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Representative internucleoside linkages having a chiral center include, but are not limited to, alkylphosphonates and phosphorothioates. Modified oligonucleotides comprising internucleoside linkages having a chiral center can be prepared as populations of modified oligonucleotides comprising stereo-random internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereo-random. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003); Wan et al., Nuc. Acid. Res. 42, 13456 (2014); Chapter 10 of Locked Nucleic Acid Aptamers in Nucleic Acid and Peptide Aptamers: Methods and Protocols v 535, 2009 by Barciszewski et al., editor Gunter Mayerand; and WO 2017/015555. In certain embodiments, a population of modified oligonucleotides is enriched for modified oligonucleotides having at least one indicated phosphorothioate in the (Sp) configuration. In another embodiment, a population of modified oligonucleotides is enriched for modified oligonucleotides having at least one indicated phosphorothioate in the (Rp) configuration.

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethylcytosine, xanthine, hypoxanthine, 2-aminoadenine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

In certain embodiments, modified sugar moieties are nonbicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2, 4, and/or 5 positions. In certain embodiments one or more non-bridging substituent of nonbicyclic modified sugar moieties is branched. Examples of 2-substituent groups suitable for non-bicyclic modified sugar moieties include but are not limited to: 2-F, 2-OCH₃ (“OMe” or “O-methyl”), and 2-O(CH₂)₂OCH₃ (“MOE”). In certain embodiments, 2-substituent groups are selected from among: halo, allyl, amino, azido, SH, CN, OCN, CF₃, OCF₃, O—C₁₋₁₀ alkoxy, O—C₁₋₁₀ substituted alkoxy, O—C₁₋₁₀ alkyl, O—C₁₋₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁₋₁₀ alkyl, and the 2-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), and alkyl. Examples of 5-substituent groups suitable for non-bicyclic modified sugar moieties include but are not limited to: 5-methyl (R or S), 5-vinyl, and 5-methoxy. In certain embodiments, non-bicyclic modified sugar moieties comprise more than one non-bridging sugar substituent, for example, 2-F-5-methyl sugar moieties and the like.

In certain embodiments, a 2′-substituted non-bicyclic modified nucleoside comprises a sugar moiety comprising a nonbridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, —O—(CH₂)ₙCH₃ (where n is 13-17, and in one preferred embodiment, 15), and N-substituted acetamide (OCH₂C(═O)—N(Rₘ)(Rₙ)), where each Rₘ and Rₙ is, independently, H, an amino protecting group, or substituted or unsubstituted C₁₋₁₀ alkyl. In one embodiment, the 2′-substituent is —O-hexadecyl (C16).

In certain embodiments, a 2′-substituted nucleoside nonbicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a nonbridging 2′-substituent group selected from: F, OCH₃, and OCH₂CH₂OCH₃. In one embodiment, the 2′-substituent is F. In another embodiment, the 2′-substituent is OCH₃ (methoxy).

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4 and the 2 furanose ring atoms. Examples of such 4 to 2 bridging sugar substituents include but are not limited to: 4-CH₂-2, 4-(CH₂)₂-2, 4-(CH₂)₃-2, 4-CH₂—O-2 (“LNA”), 4-CH₂—S-2, 4-(CH₂)₂—O-2 (“ENA”), 4-CH(CH₃)—O-2 (referred to as “constrained ethyl” or “cEt”), 4-CH₂—O—CH₂-2, 4-CH₂—N(R)-2, 4-CH(CH₂OCH₃)—O-2 (“constrained MOE” or “cMOE”) and analogs thereof, 4-C(CH₃)(CH₃)—O-2 and analogs thereof, 4-CH₂—N(OCH₃)-2 and analogs thereof, 4-CH₂—O—N(CH₃)-2, 4-CH₂—C(H)(CH₃)-2, 4-CH₂—C(═CH₂)-2 and analogs thereof, 4-C(RₐR_{b})—N(R)—O-2, 4-C(RₐR_{b})—O—N(R)-2, 4-CH₂—O—N(R)-2, and 4-CH₂—N(R)—O-2, wherein each R, Rₐ, and R_{b}, is, independently, H, a protecting group, or C₁₋₁₂ alkyl. In one embodiment, the modified sugar moiety comprises a 4-(CH₂)₂—O-2 bridge (where the “4” represents the 4 furanose ring atom and the “2” represents the 2 furanose ring atom) (“ENA” or ethylene-bridged nucleic acid).

In certain embodiments, such 4 to 2 bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently, H, a protecting group, hydroxyl, C₁₋₁₂ alkyl, substituted C₁₋₁₂ alkyl, C₁₋₁₂ alkenyl, substituted C₂₋₁₂ alkenyl, C₂₋₁₂ alkynyl, substituted C₂₋₁₂ alkynyl, C₅₋₂₀ aryl, substituted C₅₋₂₀ aryl, heterocycle radical, substituted heterocycle radical, heteroaryl, substituted heteroaryl, C₅₋₇ alicyclic radical, substituted C₅₋₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁₋₁₂ alkyl, substituted C₁₋₁₂ alkyl, C₂₋₁₂ alkenyl, substituted C₂₋₁₂ alkenyl, C₂₋₁₂ alkynyl, substituted C₂₋₁₂ alkynyl, C₅₋₂₀ aryl, substituted C₅₋₂₀ aryl, acyl (C(═O)—H), substituted acyl, a heterocycle radical, a substituted heterocycle radical, C₁₋₁₂ aminoalkyl, substituted C₁₋₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

Another modification of the ASO involves chemically linking to the ASO one or more ligands, moieties or conjugates that enhance the activity, cellular distribution or cellular uptake of the ASO. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

The disclosure provides oligonucleotides (modified or unmodified) that can be used to modulate SYF2 expression. Table 1 provides (5′ to 3′) generic sequence of bases for the SYF2 antisense oligonucleotides or inhibitory nucleic acids of the disclosure:

TABLE 1

| SEQ ID NO. | Sequence |
|---|---|
| 1 | ATAACAACCTTTCCCTCTGG |
| 2 | CCTTTCCCTCTGGAGTTTTA |
| 3 | GGAAATAAAGATGAAGCAGG |
| 4 | GCGTCAAAATAAGAGTCATG |
| 5 | GGCCATGAGTTAATGATTGT |
| 6 | GGTGTATGGAATTTCCCATA |
| 7 | CTCACCAGGGGTATTGGGTG |
| 8 | TAATCCCAAGCATTTATTGC |
| 9 | GGAACTGTACATGATCAGAT |
| 10 | GATCAGATCATGGAAGGATA |
| 11 | AGATCATGGAAGGATAGCTC |
| 12 | GTGGCCACTGCCATCTTCCT |
| 13 | TCATCACTGTCCTTAGCTTT |
| 14 | TGTCCTTAGCTTTGGAGTAC |
| 15 | GTACACAACTCCAAGTGGCC |
| 16 | CAAATTCCACACTGATAGCA |
| 17 | CCACACTGATAGCAACAATG |
| 18 | GACTGCATCTGATGTGTGCT |
| 19 | GCTGGCAATCTTAAGCCCAA |
| 20 | GCTGAGTGAGAAGGCATGGA |
| 21 | GAGTGAGAAGGCATGGACTA |
| 22 | CATGGACTACTAAATTCTGG |
| 23 | TTACAAAACAACTTCACTAC |
| 24 | CGCCATCACAACCTTTCTCT |
| 25 | CTAGCAGAAATTTTTACCCC |
| 26 | CCCATAGAATCTTTCAGCTT |
| 27 | CCAGATCTATGACCATCCTG |
| 28 | GATCTATGACCATCCTGTCA |
| 29 | CCATCCTGTCAATTTCCTCT |
| 30 | ATGAAGAAGACTATTGGATG |
| 31 | CTCTTCTCCATGTTTTTCTC |
| 32 | GTTGGGAAAAACTCTTCTCC |
| 33 | GTTTCCATGTCAGGTTTGAT |
| 34 | CCATGTCAGGTTTGATCTGC |
| 35 | GATGATACTGGCGTAACTGG |
| 36 | GGCAGCAGCATAATCTGAAA |
| 37 | CCAGCAACTTCACTTTCTCA |
| 38 | GCATCTTCTGCACTGATCTC |
| 39 | TAGTCTTCTCCTCTTGCCGC |
| 40 | CTCATAGTCTTCTCCTCTTG |
| 41 | TTCCCACTTCCGGCAACAAG |
| 42 | CTCTTCCCACTTCCGGCAAC |
| 43 | AGCCGCCATCACAACCTTTC |
| 44 | GTATATGCGTCAAAATAAGA |
| 45 | GTTAGAAATTCTAATCCCAA |
| 46 | TACATCTTATATCCAAGCAC |
| 47 | GGATTAGACAGCTGTTCCTC |
| 48 | GATATCTGCATCATCATTAT |
| 49 | TCTGCATCATCATTATAAGG |
| 50 | GTAACTGGGCAGCAGCATAA |
| 51 | TAACAACCTTTCCCTCTGGA |
| 52 | AGGCATGGACTACTAAATTC |
| 53 | GAAGGCATGGACTACTAAAT |
| 54 | CTGAGTGAGAAGGCATGGAC |
| 55 | TGAGTGAGAAGGCATGGACT |
| 56 | AGAAGGCATGGACTACTAAA |
| 57 | GAGAAGGCATGGACTACTAA |
| 58 | TGAGAAGGCATGGACTACTA |
| 59 | GTGAGAAGGCATGGACTACT |
| 60 | AGTGAGAAGGCATGGACTAC |
| 61 | AATCTTTCAGCTTTCTTGTT |
| 62 | AGAATCTTTCAGCTTTCTTG |
| 63 | CTACTAAATTCTGGATTACT |
| 64 | CACACTGATAGCAACAATGA |
| 65 | ACACTGATAGCAACAATGAC |
| 66 | CACTGATAGCAACAATGACT |
| 67 | ACTGATAGCAACAATGACTG |
| 68 | CTGATAGCAACAATGACTGC |
| 69 | TGATAGCAACAATGACTGCA |
| 70 | GGCAATCTTAAGCCCAAAAT |
| 71 | GCAATCTTAAGCCCAAAATG |
| 72 | CAATCTTAAGCCCAAAATGC |
| 73 | ATGCTTCAAAGATTAAACAG |
| 74 | CTTCAAAGATTAAACAGCCA |
| 75 | CATCTTATATCCAAGCACAA |
| 76 | ATCTTATATCCAAGCACAAA |
| 77 | CTTATATCCAAGCACAAAAA |
| 78 | CATGAAAGGATATACGTTTA |
| 79 | ATTTCTAAATGCTGAGTGAG |
| 80 | CTAAATGCTGAGTGAGAAGG |
| 81 | TAAATGCTGAGTGAGAAGGC |
| 82 | AAATGCTGAGTGAGAAGGCA |
| 83 | AATGCTGAGTGAGAAGGCAT |
| 84 | ATGCTGAGTGAGAAGGCATG |
| 85 | TGCTGAGTGAGAAGGCATGG |
| 86 | CATGAAGAAGACTATTGGAT |
| 87 | TACACAACTCCAAGTGGCCC |
| 88 | ACACAACTCCAAGTGGCCCG |
| 89 | CATCACTGTCCTTAGCTTTG |
| 90 | ATCACTGTCCTTAGCTTTGG |
| 91 | TCACTGTCCTTAGCTTTGGA |
| 92 | CACTGTCCTTAGCTTTGGAG |
| 93 | ACTGTCCTTAGCTTTGGAGT |
| 94 | CTGTCCTTAGCTTTGGAGTA |
| 95 | GATCATGGAAGGATAGCTCC |
| 96 | ATCATGGAAGGATAGCTCCA |
| 97 | TCATGGAAGGATAGCTCCAC |
| 98 | CTCCCCGACCTTGGTCACAG |
| 99 | ATGGAAGGATAGCTCCACTC |
| 100 | GGAAGGATAGCTCCACTCCT |
| 101 | CAGCCCAGTCAATAAGGTTC |
| 102 | GCCTCTATTGAGTTGTGTGG |
| 103 | TATCAACTGGCTGAACTACC |
| 104 | ATCAACTGGCTGAACTACCA |
| 105 | GTCAGGTTTGATCTGCTTGG |
| 106 | AACCCCAGCACTAAAGCATT |
| 107 | GGACTCTTATCCCTAATACA |
| 108 | TCCCTATCAACTGGCTGAAC |
| 109 | GCTGGACTCTTATCCCTAAT |
| 110 | ACAGTAGTTTAGGTGTCACC |
| 111 | GACTAACAGCCCAGTCAATA |
| 112 | ACTACATGAGGCACCCCCTG |
| 113 | GAGAGGCTCCAGGACTTATC |
| 114 | TTGCTGGACTCTTATCCCTA |
| 115 | GGTTGTTAGTAATGGAGACT |
| 116 | GGTTTGATCTGCTTGGTCAA |
| 117 | TGTTTGGACCTTCCTATTGA |
| 118 | TTAGGTCATAAGCTGGTGGC |
| 119 | ATCCCTAGTATATTCTTGGC |
| 120 | CTACCATCCCTAGTATATTC |
| 121 | GGTCAGGCAGGACATATTAA |

In one embodiment, the disclosure provides modified oligonucleotides consisting of 12-30 linked nucleosides and having a nucleobase sequence comprising at least 8, at least 9, at least 10, at least 11 at least 12, at least 13, at least 14, at least 15, at least 16, at least 17, at least 18, at least 19 or at least 20 consecutive nucleotide bases of any of the nucleobase sequences of SEQ ID NO:1-121 in Table 1. In some embodiments, the modified oligonucleotide is at least 80% to 100% (i.e., 80%, 82%, 84%, 86%, 88%, 90%, 92%, 94%, 96%, 98% or 100%>; or any numerical range or value between any of the foregoing values) identical to any of the sequences comprising or consisting of SEQ ID NO:1-121.

The sequences provided in Table 1 can be used to design antisense molecules for inhibition of SYF2 expression. For example, gapmer oligonucleotides can be designed using the sequences in Table 1 and can comprise a 5′-wing of about 3-5 nucleotides, a 3′-wing of about 3-5 nucleotides and a gap region comprising 8-12 consecutive deoxyribonucleosides of any one of the sequences of Table 1. In one embodiment, an oligonucleotide of the disclosure comprises a gapmer having a gap segment of at least 8, at least 9, at least 10, at least 11 at least 12, at least 13, at least 14, at least 15, at least 16, at least 17, at least 18, at least 19 or at least 20 consecutive nucleotide bases of any of the nucleobase sequences of SEQ ID NO:1-121 in Table 1; flanked by a 5′ and 3′ wing segments, wherein the gap segment is located between the 5′ and 3′ wing segments and wherein each of the wing segments comprises a modified sugar. In one embodiment, the gap segment is 8-10 nucleosides in length and each wing segment is 3-5 modified nucleosides in length. In yet another embodiment, an oligonucleotide of the disclosure comprises a 5′ wing segment comprising modified sugars and having the nucleobase sequence of the first 3-5 nucleobases of any of SEQ ID NO:1-121, followed by a gap of the next 8-12 unmodified nucleotides of the same sequence corresponding to SEQ ID NO:1-121, followed by a 3′ wing segment comprising modified sugars and having the nucleobase sequence of the last 3-5 nucleobases of the same sequence corresponding to SEQ ID NO:1-121. Table 2 provides MOE gapmers of the disclosure.

The 5′ and/or 3′ wings can comprise the following chemistries: 2′-OMe, 2′-MOE, LNA or DNA, by themselves or used in combination with one another. The backbone linkage of the 5′ and/or 3′ wings can be phosphorothioate or a mixture of phosphodiester and phosphorothioate. Linkages in the gap region can be phosphorothioate.

In some embodiments, the oligonucleotide is single stranded. In some embodiments the oligonucleotide comprises or is complexed with a moiety that neutralizes charge on the oligonucleotide to promote uptake and transfer across a cell membrane.

In one embodiment, each of the ASOs in Table 1 has the following 5-10-5 motif: 2MOE*2MOE-2MOE-2MOE-2MOE-N*N*N*N*N*N*N*N*N*N*2MOE-2MOE-2MOE*2MOE*2MOE where (i) 2MOE is a nucleobase with a 2′-OCH₂CH₂—OCH₃ group (i.e., 2′-MOE), (ii) N is a nucleobase, (iii) the asterisk (*) refers to a phosphorothioate linkage, and (iv) the dash (—) refers to a phosphodiester linkage. Table 2 below shows this motif on SEQ ID NOs. 1-20 and 102-121 (here SEQ ID NOs. 122-141 and 142-161).

Table 2: The Sequence of Bases in SYF2 Antisense Oligonucleotides—(ASOs). (Gapmer design: 5′-five 2′-methoxyethylribose nucleotides-ten DNA nucleotides-five 2′-methoxyethylribose nucleotides-3′; Capital letters are 2′-methoxyethylribose nucleosides; lower case are DNA nucleosides; asterisks (*) are phosphorothioate linkages; linkages which do not have an asterisk are phosphodiester linkages) (Note that the following table provides 2′MOE wings; however, alternative wings comprising 2′-OMe or LNA (locked nucleic acid) are contemplated).

TABLE 2

| SEQ ID NO. | Sequence |
|---|---|
| 122 | A*TAACa*a*c*c*t*t*t*c*c*c*TCT*G*G |
| 123 | C*CTTTc*c*c*t*c*t*g*g*a*g*TTT*T*A |
| 124 | G*GAAAt*a*a*a*g*a*t*g*a*a*GCA*G*G |
| 125 | G*CGTCa*a*a*a*t*a*a*g*a*g*TCA*T*G |
| 126 | G*GCCAt*g*a*g*t*t*a*a*t*g*ATT*G*T |
| 127 | G*GTGTa*t*g*g*a*a***t*t*c*CCA*T*A |
| 128 | C*TCACc*a*g*g*g*g*t*a***t*GGG*T*G |
| 129 | T*AATCc*c*a*a*g*c*a*t*t***ATT*G*C |
| 130 | G*GAACt*g*t*a*c*a*t*g*a*t*CAG*A*T |
| 131 | G*ATCAg*a*t*c*a*t*g*g*a*a*GGA*T*A |
| 132 | A*GATCa*t*g*g*a*a*g*g*a*t*AGC*T*C |
| 133 | G*TGGCc*a*c*t*g*c*c*a*t*c*TTC*C*T |
| 134 | T*CATCa*c*t*g*t*c*c*t*t*a*GCT*T*T |
| 135 | T*GTCCt*t*a*g*c*t*t*t*g*g*AGT*A*C |
| 136 | G*TACAc*a*a*c*t*c*c*a*a*g*TGG*C*C |
| 137 | C*AAATt*c*c*a*c*a*c*t*g*a*TAG*C*A |
| 138 | C*CACAc*t*g*a*t*a*g*c*a*a*CAA*T*G |
| 139 | G*ACTGc*a*t*c*t*g*a*t*g*t*GTG*C*T |
| 140 | G*CTGGc*a*a*t*c*t*t*a*a*g*CCC*A*A |
| 141 | G*CTGAg*t*g*a*g*a*a*g*g*c*ATG*G*A |
| 142 | G*CCTCt*a*t*t*g*a*g*t*t*g*TGT*G*G |
| 143 | T*ATCAa*c*t*g*g*c*t*g*a*a*CTA*C*C |
| 144 | A*TCAAc*t*g*g*c*t*g*a*a*c*TAC*C*A |
| 145 | G*TCAGg*t*t*t*g*a*t*c*t*g*CTT*G*G |
| 146 | A*ACCCc*a*g*c*a*c*t*a*a*a*GCA*T*T |
| 147 | G*GACTc*t*t*a*t*c*c*c*t*a*ATA*C*A |
| 148 | T*CCCTa*t*c*a*a*c*t*g*g*c*TGA*A*C |
| 149 | G*CTGGa*c*t*c*t*t*a*t*c*c*CTA*A*T |
| 150 | A*CAGTa*g*t*t*t*a*g*g*t*g*TCA*C*C |
| 151 | G*ACTAa*c*a*g*c*c*c*a*g*t*CAA*T*A |
| 152 | A*CTACa*t*g*a*g*g*c*a*c*c*CCC*T*G |
| 153 | G*AGAGg*c*t*c*c*a*g*g*a*c*TTA*T*C |
| 154 | T*TGCTg*g*a*c*t*c*t*t*a*t*CCC*T*A |
| 155 | G*GTTGt*t*a*g*t*a*a*t*g*g*AGA*C*T |
| 156 | G*GTTTg*a*t*c*t*g*c*t*t*g*GTC*A*A |
| 157 | T*GTTTg*g*a*c*c*t*t*c*c*t*ATT*G*A |
| 158 | T*TAGGt*c*a*t*a*a*g*c*t*g*GTG*G*C |
| 159 | A*TCCCt*a*g*t*a*t*a*t*t*c*TTG*G*C |
| 160 | C*TACCa*t*c*c*c*t*a*g*t*a*TAT*T*C |
| 161 | G*GTCAg*g*c*a*g*g*a*c*a*t*ATT*A*A |

The SYF2 antisense or inhibitory nucleic acids of the disclosure can inhibit the expression and thus the activity associate with SYF2. The SYF2 antisense or inhibitory nucleic acids can include any combination of the oligonucleotides set forth in Table 2 and sequences that are 98%-99% identical thereto.

### Treatment Methods

Yet another embodiment is a method of treating a subject having a SYF2 disease or disorder by administering a therapeutically effective amount of a SYF2 ASO or a pharmaceutical composition described herein.

One embodiment is a method of treating a subject having a neurological or neurodegenerative disease by administering a therapeutically effective amount of a SYF2 ASO or a pharmaceutical composition described herein. The neurological disease may be a neurodegenerative disease. The neurodegenerative disease may result in motor neuron degeneration, for example. The neurological disease may be amyotrophic lateral sclerosis (ALS), Huntington's disease, Alzheimer's disease, or frontotemporal dementia, for example. Further examples of neurological diseases include, but are not limited to, Parkinson's disease, multiple sclerosis, peripheral myopathy, Rasmussen's encephalitis, attention deficit hyperactivity disorder, autism, central pain syndromes, anxiety, and/or depression, for example.

The neurological disease may be associated with aberrant endosomal trafficking. For example, endosomal pathways and endosomes are necessary components for the recycling or breakdown of membrane-bound proteins, trafficking of Golgi-associated proteins, and the extracellular release of proteins in exosomes. These processes aid neurotransmission and drive a balance between recycling and degradation of synaptic vesicles or neurotransmitter receptors, for example.

The neurological disease may be associated with aberrant lysosome degradation. Alterations in the lysosome degradation may be present in the neurological disease, such as a neurodegenerative disease. Cathepsin imbalance during aging and age-related diseases may provoke deleterious effects on central nervous system (CNS) neurons and lysosomes may be sites for the unfolding and partial degradation of membrane proteins or their precursors that subsequently become expelled from a cell, or are released from dead cells and accumulate as pathological entities.

A health care professional may diagnose a subject as having a disease associated with motor neuron degeneration by the assessment of one or more symptoms of motor neuron degeneration. To diagnose a neurological disease, a physical exam may be followed by a thorough neurological exam. The neurological exam may assess motor and sensory skills, nerve function, hearing and speech, vision, coordination and balance, mental status, and changes in mood or behavior. Non-limiting symptoms of a disease associated with a neurological disease may be weakness in the arms, legs, feet, or ankles; slurring of speech; difficulty lifting the front part of the foot and toes; hand weakness or clumsiness; muscle paralysis; rigid muscles; involuntary jerking or writing movements (chorea); involuntary, sustained contracture of muscles (dystonia); bradykinesia; loss of automatic movements; impaired posture and balance; lack of flexibility; tingling parts in the body; electric shock sensations that occur with movement of the head; twitching in arm, shoulders, and tongue; difficulty swallowing; difficulty breathing; difficulty chewing; partial or complete loss of vision; double vision; slow or abnormal eye movements; tremor; unsteady gait; fatigue; loss of memory; dizziness; difficulty thinking or concentrating; difficulty reading or writing; misinterpretation of spatial relationships; disorientation; depression; anxiety; difficulty making decisions and judgments; loss of impulse control; difficulty in planning and performing familiar tasks; aggressiveness; irritability; social withdrawal; mood swings; dementia; change in sleeping habits; wandering; and change in appetite.

Tests may be performed to rule diseases and disorders that may have symptoms similar to those of neurological diseases, measure muscle involvement, assess neuron degeneration. Non-limiting examples of tests are electromyography (EMG); nerve conduction velocity study; laboratory tests of blood, urine, or other substances; magnetic resonance imaging (MRI); magnetic resonance spectroscopy; muscle or nerve biopsy; transcranial magnetic stimulation; genetic screening; x-rays; fluoroscopy; angiography; computed tomography (CT); positron emission tomography; cerebrospinal fluid analysis; intrathecal contrast-enhanced CT scan; electroencephalography; electronystagmography; evoked response; polysomnogram; thermography; and ultrasound. A health care professional may also assess the patient's family history of diseases associated with motor neuron degeneration and make a diagnosis in part based on a familial history of neurological diseases. A healthcare professional may diagnose a disease associated with neurological disease in a subject after the presentation of one or more symptoms.

Neurodegenerative diseases result in the progressive destruction of neurons that affects neuronal signaling. For example, a neurodegeneration may be amyotrophic lateral sclerosis, Alzheimer's disease, Huntington's disease, Friedreich's ataxia, Lewy body disease, Parkinson's disease, spinal muscle atrophy, primary lateral sclerosis, progressive muscle atrophy, progressive bulbar palsy, and pseudobulbar palsy.

Diseases associated with motor neuron degeneration may be a condition that results in the progressive destruction of motor neurons that interferes with neuronal signaling to the muscles, leading to muscle weakness and wasting. In healthy individuals, upper motor neurons transmit signals from the brain to lower motor neurons in the brain stem and spinal cord, which then transmit the signal to the muscles to result in voluntary muscle activity. The destruction of upper and lower motor neurons affects activity such as breathing, talking, swallowing, and walking, and overtime these functions can be lost. Examples of motor neuron diseases include, but are not limited to, amyotrophic lateral sclerosis, primary lateral sclerosis, progressive muscle atrophy, progressive bulbar palsy, and pseudobulbar palsy.

Neuronal hyperexcitability may occur when receptors for the excitatory neurotransmitter glutamate (glutamate receptors) such as the NMDA receptor and AMPA receptor are over-activated by excess glutamate or by other compounds or neurotransmitters acting on the glutamate receptors. Excitotoxicity may result from neuronal hyperexcitability. Excitotoxicity is the pathological process by which nerve cells are damaged or killed by excessive stimulation. The excessive stimulation allows high levels of calcium ions (Ca²⁺) to enter the cell. Ca²⁺ influx into cells activates a number of enzymes, including phospholipases, endonucleases, and proteases such as calpain. These enzymes can damage cell structures such as components of the cytoskeleton, membrane, and DNA.

Neuronal hyperexcitability may be involved in spinal cord injury, stroke, traumatic brain injury, hearing loss (through noise overexposure or ototoxicity), epilepsy, painful neuropathies, attention deficit hyperactivity disorder, autism, central pain syndromes, neurodegenerative diseases, multiple sclerosis, Alzheimer's disease, amyotrophic lateral sclerosis (ALS), Parkinson's disease, frontotemporal dementia, schizophrenia, Rasmussen's encephalitis, Huntington's disease, alcoholism or alcohol withdrawal and especially over-rapid benzodiazepine withdrawal, and also Huntington's disease. Other common conditions that cause excessive glutamate concentrations around neurons are hypoglycemia. Blood sugars are the primary glutamate removal method from inter-synaptic spaces at the NMDA and AMPA receptor site.

The herein described methods of treatment may comprise administering to a subject in need thereof a composition comprising an effective amount of one or more antisense oligonucleotides that treats neurological diseases by inhibiting SYF2 expression. The one or more antisense oligonucleotides may decrease or inhibit neurodegeneration. The one or more antisense oligonucleotides may decrease neuronal hyperexcitability.

The composition may inhibit kinase activity by inhibiting expression of a kinase. The composition may inhibit SYF2 activity or expression. The one or more antisense oligonucleotides can be combined with small molecule therapeutic agents (such as apilimod and/or YM201636).

Methods of treatment may include any number of modes of administering a disclosed composition. Modes of administration may include aqueous, lipid, oily or other solutions, solutions in simulated cerebrospinal fluid, emulsions such as oil-in-water emulsions, liposomes, aqueous or oily suspensions and the like. Typically, an ASO of the disclosure will be administered directly to the CNS of the subject. Accordingly, the formulation or composition will be sterile and more preferably be suitable for injection. The following formulations and methods are merely exemplary and are in no way limiting.

Formulations suitable for parenteral administration include aqueous and non-aqueous, isotonic sterile injection solutions, which may contain anti-oxidants, buffers, bacteriostats, and solutes that render the formulation isotonic with the blood of the intended recipient, and aqueous and non-aqueous sterile suspensions that may include suspending agents, solubilizers, thickening agents, stabilizers, and preservatives. The formulations may be presented in unit-dose or multi-dose sealed containers, such as ampules and vials, and may be stored as liquids or in a freeze-dried (lyophilized) condition requiring only the addition of the sterile liquid excipient, for example, water for injections, immediately prior to use. Extemporaneous injection solutions and suspensions may be prepared from sterile powders, granules, and tablets. The formulation may be provided in a pre-filled syringe.

Additional therapeutic agent(s) may be administered simultaneously or sequentially with the disclosed one or more antisense or inhibitory nucleic acids and compositions. Sequential administration includes administration before or after the disclosed one or more antisense or inhibitory nucleic acids or compositions. In some embodiments, the additional therapeutic agent or agents may be administered in the same composition as the disclosed one or more antisense or inhibitory nucleic acids. In other embodiments, there may be an interval of time between administration of the additional therapeutic agent and the disclosed one or more antisense or inhibitory nucleic acids. In some embodiments, administration of an additional therapeutic agent with a disclosed one or more antisense or inhibitory nucleic acids may allow lower doses of the other therapeutic agents and/or administration at less frequent intervals. When used in combination with one or more other active ingredients, the one or more antisense or inhibitory nucleic acids of the disclosure and the other active ingredients may be used in lower doses than when each is used singly. Accordingly, the pharmaceutical compositions of the disclosure include those that contain one or more other active ingredients, in addition to one or more antisense or inhibitory nucleic acids of the disclosure. The above combinations include combinations of one or more antisense or inhibitory nucleic acids of the disclosure not only with one other active compound, but also with two or more other active compounds. For example, the compound of the disclosure may be combined with a variety of drugs to treat neurological diseases. The antisense oligonucleotide may be covalently linked to another oligonucleotide, such as one with a target other than SYF2. The antisense oligonucleotide may be covalently linked to an antibody.

The disclosed one or more antisense or inhibitory nucleic acids can be combined with the following, but are not limited, anticholinergic drugs, anticonvulsants, antidepressants, benzodiazepines, decongestants, muscle relaxants, pain medications, and/or stimulants. Additional types of therapy and treatment include, but are not limited to digital communication devices, feeding tubes, mechanical ventilation, nutritional support, deep brain stimulation, occupational therapy, physical therapy, and/or speech therapy.

The disclosed composition(s) may be incorporated into a pharmaceutical composition suitable for administration to a subject (such as a patient, which may be a human or non-human). The pharmaceutical compositions may comprise a carrier (e.g., a pharmaceutically acceptable carrier). Any suitable carrier can be used within the context of the disclosure, and such carriers are well known in the art. The choice of carrier will be determined, in part, by the particular use of the composition (e.g., administration to an animal) and the particular method used to administer the composition. Accordingly, there is a wide variety of suitable formulations of the composition of the present invention.

The pharmaceutical compositions may include a therapeutically effective amount or a prophylactically effective amount of the antisense oligonucleotide. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of one or more antisense or inhibitory nucleic acids of the disclosure are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions may include one or more pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogenfree water; isotonic saline; Ringer's solution; ethyl alcohol, and phosphate buffer solutions, as well as other non-toxic compatible lubricants such as, but not limited to, sodium lauryl sulfate and magnesium stearate, as well as releasing agents, coating agents, preservatives and antioxidants may also be present in the composition, according to the judgment of the formulator.

The route by which the disclosed one or more antisense or inhibitory nucleic acids are administered, and the form of the composition will dictate the type of carrier to be used.

The pharmaceutical compositions of the disclosure can be administered in a number of ways depending upon whether local or systemic treatment is desired and upon the area to be treated. Administration can be parenteral including intravenous, intraarterial, subcutaneous, intraperitoneal or intramuscular injection or infusion; or intracranial, e.g., intrathecal, intracerebroventricular, or intraventricular, administration. In one embodiment the antisense or inhibitory nucleic acid is administered by intravenous, intraperitoneal, or as a bolus injection or administered directly into the target organ. In another embodiment, the antisense or inhibitory nucleic acid is administered intrathecally or intra-cerebroventricular as a bolus injection.

Carriers for systemic administration typically include at least one of solvent, diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmelose, crospovidone, sodium carboxymethyl starch, sodium starch glycolate, clays, and ion exchange resins. The amount of disintegrant(s) in a systemic composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Compositions and formulations for parenteral, intrathecal, intra-cerebroventricular, or intraventricular administration can include sterile aqueous solutions which can also contain buffers, diluents and other suitable additives such as, but not limited to, penetration enhancers, carrier compounds and other pharmaceutically acceptable carriers or excipients. For example, an intrathecal cerebrospinal fluid (CSF) catheter can be used to deliver antisense formulations of the disclosure. The catheter can be inserted at the L3 or L4 vertebrae. The distal tip of the catheter extends within the intrathecal space to approximately the L1 vertebrae. Antisense oligonucleotides are dissolved in saline, are sterilized by filtration, and are administered at 0.33 ml/min in a 1.0 ml volume followed by a 0.5 ml sterile water flush. Total infusion time is 4.5 min.

Compositions for parenteral administration typically include 0.1% to 10% of actives and 90% to 99.9% of a carrier including a diluent and a solvent.

The amount of the carrier employed in conjunction with a disclosed compound is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

In vivo testing of candidate antisense or inhibitory nucleic acids may be conducted by means known to one of ordinary skill in the art. For example, the candidate one or more antisense or inhibitory nucleic acids may be administered to a mammal, such as a mouse or a rabbit. The mammal may be administered, by any route deemed appropriate, a dose of a candidate antisense or inhibitory nucleic acids. Conventional methods and criteria can then be used to monitor animals for signs of reduction or improvement of motor neuron activity and/or expression or activity of SYF2 gene or protein, respectively. If needed, the results obtained in the presence of the candidate antisense or inhibitory nucleic acids can be compared with results in control animals that are not treated with the candidate antisense or inhibitory nucleic acids. Dosing studies may be performed in, or in conjunction with, the herein described methods for identifying one or more antisense or inhibitory nucleic acids capable of treating a neurological disease and/or any follow-on testing of candidate antisense or inhibitory nucleic acids in vivo. One of skill in the art of medicine may determine the appropriate dosage of one or more antisense or inhibitory nucleic acids. The dosage may be determined by monitoring the subject for signs of disease inhibition or amelioration. The dosage may be increased or decreased to obtain the desired frequency of treatment. The toxicity and efficacy of one or more antisense or inhibitory nucleic acids may be determined by standard pharmaceutical procedures in cell cultures or experimental animals, e.g. determining the lethal dose to 50% of the population (LD50) and the dose therapeutically effective in 50% of the population (ED50). The dose ratio of LD50/ED50 is the therapeutic index and, indicating the ratio between the toxic and therapeutic effects. A delivery system may be designed to help prevent toxic side effects, by delivering the one or more antisense or inhibitory nucleic acids to specific targets, e.g., delivered specifically to motor or central nervous system neurons. The optimal dose of the one or more antisense or inhibitory nucleic acids may be determined based on results of clinical electrophysiology or electromyography to analyze excitability in peripheral nerves, for example.

The dosage for use in humans may be determined by evaluating data obtained from animal studies and cell culture assays. The preferred dosage will have little or no toxicity and include the ED50. The dosage may vary depending on the dosage form and route of administration. For any antisense or inhibitory nucleic acid used in the methods described herein, the dosage may be estimated initially in cell culture. A dose may be formulated in animal models that includes the concentration of the test compound which achieves a half maximal inhibition of symptoms (LD50) as determined in cell culture. Such information obtained from cell cultures and animal models may be used to more accurately determine useful doses in humans.

In another embodiment, an antisense oligonucleotide of the present invention is expressed from a transgene, e.g., as an antisense RNA transcript. A transgene may be administered to a subject in a DNA expression construct that is engineered to express an antisense RNA transcript in a subject. A DNA expression construct may be administered directly or using a viral vector (e.g., a recombinant AAV (rAAV) vector) or other suitable vector. Viral vectors that have been used for gene therapy protocols include, but are not limited to, retroviruses, other RNA viruses such as poliovirus or Sindbis virus, adenovirus, adeno-associated virus (AAV), herpes viruses, SV 40, vaccinia, lentivirus and other DNA viruses.

The present invention has multiple aspects, illustrated by the following non-limiting examples.

### Examples

ASOs are an attractive therapeutic option for neurodegenerative diseases because of their ease of delivery to the central nervous system and their relatively low exposure to the periphery. These properties maximize target engagement in the central nervous system and minimize undesired target engagement or off-target effects in the periphery.

The disclosure provides novel antisense oligonucleotide (ASO) sequences targeting the SYF2 gene that can suppress SYF2 expression in human cells. SYF2 ASOs can also rescue the survival of motor neurons derived from sporadic ALS patients, while also rescuing TDP-43 mislocalization, neurodegeneration, and NMJ loss and resulting in improved motor function.

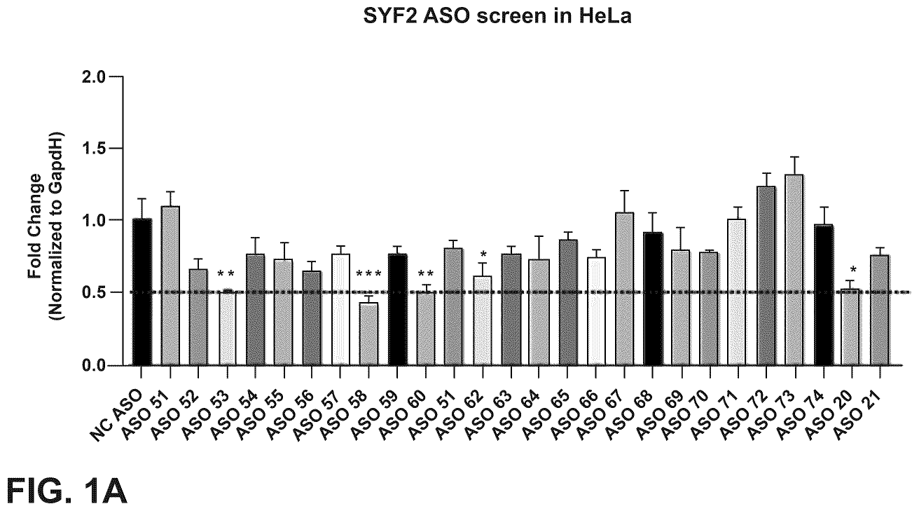

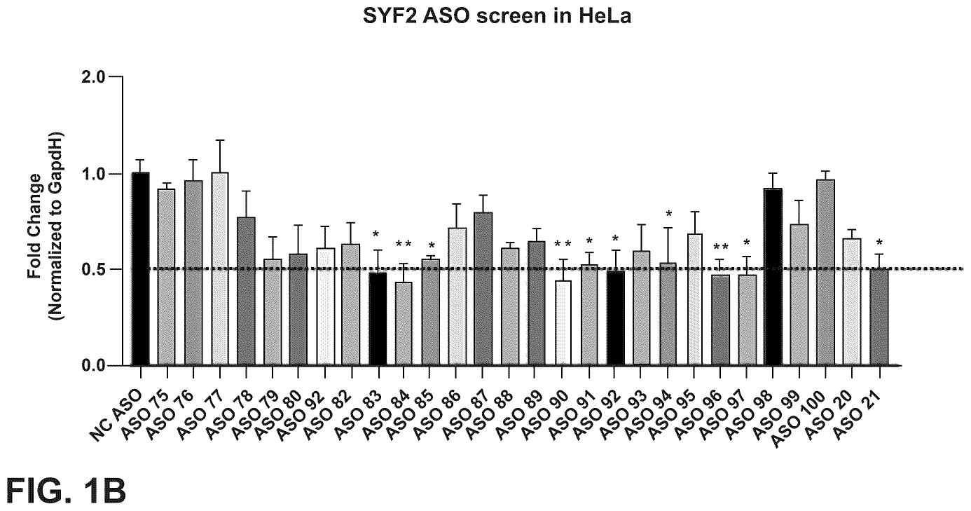

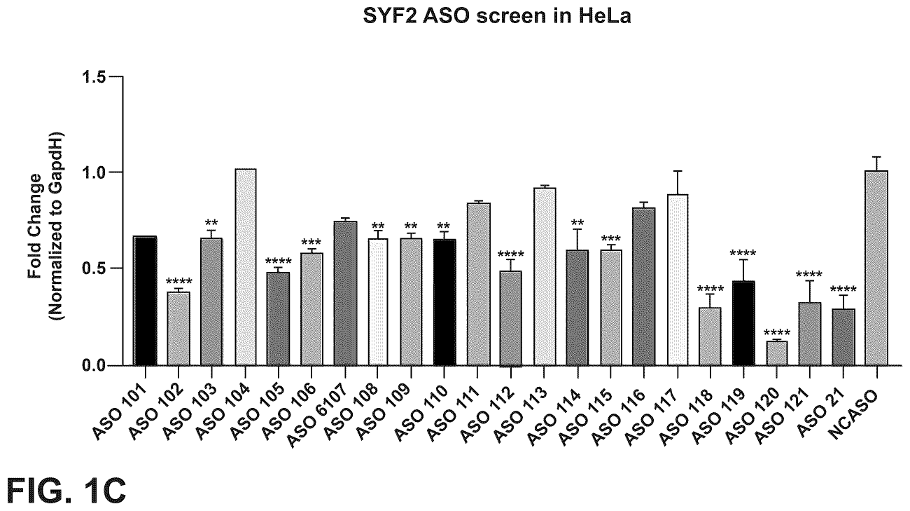

To identify ASO sequences that suppress SYF2 expression in human cells, ASOs were designed (see, Table 2) and synthesized as MOE gapmers, which contains sugar and linkage modifications that increase nuclease resistance and melting temperature while maintaining the ability to be used as a substrate of RNase H. Together, these results, as shown in FIGS. 1A-1C, show that several SYF2 ASOs suppress SYF2 expression in human cells (HeLa). As a control, NCASO was used, and the values were normalized to GapdH. FIG. 1A shows a SYF2 ASO screen in HeLa cells, measuring the fold change normalized to GadpH of ASO 50-74 (SEQ ID NOs. 50-74). FIG. 1B shows a SYF2 ASO screen in HeLa cells, measuring the fold change normalized to GadpH of ASO 75-100 (SEQ ID NOs. 75-100). FIG. 1C shows a SYF2 ASO screen in HeLa cells, measuring the fold change normalized to GadpH of ASO 101-121 (SEQ ID NOs. 101-121).

The disclosure thus provides ASOs that suppress SYF2 expression in human cells. The accompanying data suggest that these ASOs may be capable of preventing neurodegeneration in ALS and FTD patients.

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention. All references cited herein are incorporated by reference.

1. A single stranded antisense oligonucleotide that suppresses the expression of a SYF2, wherein the antisense oligonucleotide has a nucleobase sequence that comprises at least 12 or 15 consecutive nucleobases of any of the nucleobase sequences of SEQ ID NOs: 1-121.

2. The antisense oligonucleotide of claim 1, wherein the antisense oligonucleotide has a nucleobase sequence of any one of SEQ ID NOs: 1-161.

3. The antisense oligonucleotide of claim 1, wherein the antisense oligonucleotide has 18 to 20 linked nucleosides.

4. The antisense oligonucleotide of claim 1, wherein at least one internucleoside linkage is a modified internucleoside linkage.

5. The antisense oligonucleotide of claim 4, wherein at least one modified internucleoside linkage is a phosphorothioate internucleoside linkage.

6. The antisense oligonucleotide of claim 4, wherein each modified internucleoside linkage is a phosphorothioate internucleoside linkage.

7. The antisense oligonucleotide of claim 1, wherein at least one internucleoside linkage is a phosphodiester internucleoside linkage.

8. The antisense oligonucleotide of claim 7, wherein at least one internucleoside linkage is a phosphorothioate linkage and at least one internucleoside linkage is a phosphodiester linkage.

9. The antisense oligonucleotide of claim 1, wherein at least one nucleoside comprises a modified nucleobase.

10. The antisense oligonucleotide of claim 9, wherein the modified nucleobase is a 5-methylcytosine.

11. The antisense oligonucleotide of claim 1, wherein at least one nucleoside of the antisense oligonucleotide comprises a modified sugar moiety.

12. The antisense oligonucleotide of claim 11, wherein the modified sugar moiety comprises a 2′-O-methoxyethyl group.

13. The antisense oligonucleotide of claim 11, wherein the modified sugar moiety comprises a 2′-F.

14. The antisense oligonucleotide of claim 11, wherein the modified sugar moiety comprises a 2′-O-hexadecyl.

15. The antisense oligonucleotide of claim 1, wherein the antisense oligonucleotide is a gapmer.

16. The antisense oligonucleotide of claim 15, wherein the antisense oligonucleotide comprises:
a gap segment consisting of 8 to 12 linked deoxynucleosides;
a 5′ wing segment consisting of 3 to 5 linked nucleosides; and
a 3′ wing segment consisting of 3 to 5 linked nucleosides,
wherein the gap segment is positioned between the 5′ wing segment and the 3′ wing segment and wherein a nucleoside of each wing segment comprises a modified sugar moiety.

17. The antisense oligonucleotide of claim 16, wherein each nucleoside of each wing segment comprises a modified sugar moiety.

18. The antisense oligonucleotide of claim 16, wherein the nucleosides making up each wing segment comprises at least two different modified sugar moieties.

19. The antisense oligonucleotide of claim 16, wherein the nucleosides making up each wing segment comprises the same modified sugar moiety.

20. The antisense oligonucleotide of claim 17, wherein the modified sugar moiety comprises a 2′-O-methoxyethyl group.

21. The antisense oligonucleotide of claim 1, wherein the antisense oligonucleotide comprises 15 to 50 nucleosides.

22. A pharmaceutical composition comprising the antisense oligonucleotide of claim 1, and a pharmaceutically acceptable carrier, diluent and/or excipient.

23. The pharmaceutical composition of claim 22, wherein the pharmaceutical composition is formulated for parenteral delivery.

24. The pharmaceutical composition of claim 22, wherein the pharmaceutical composition is formulated for intracerebroventricular injection.

25. A method of treating a subject having a neurological or neurodegenerative disease in need thereof comprising administering a therapeutically effective amount of the pharmaceutical composition of claim 22.

26. The method of claim 25, wherein the neurological disease is associated with neuronal hyperexcitability.

27. The method of claim 25, wherein the neurological disease is associated with aberrant endosomal trafficking.

28. The method of claim 25, wherein the neurological disease is associated with aberrant lysosomal trafficking.

29. The method of claim 25, wherein the neurological disease is selected from the group consisting of familial and sporadic amyotrophic lateral sclerosis (ALS), familial and sporadic frontotemporal dementia (FTD), progressive supranuclear palsy, Alzheimer's disease, chronic traumatic encephalopathy, Parkinson's disease, Charcot Marie Tooth 2A and 4B, Huntington's disease, dementia, transmissible spongiform encephalopathy, spinobulbar muscular atrophy, dentatorubral-pallidoluysian atrophy, spinocerebellar ataxias, and Creutzfeldt-Jakob disease.

30. An oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or at least 20 consecutive nucleobases of any of the nucleobase sequences of SEQ ID NOs: 1-121.
